(19) United States
(12) Patent Application Publication
(10) Pub. No.: US 2015/0182173 A1
(43) Pub. Date: Jul. 2, 2015

(54) IMAGE DIAGNOSTIC APPARATUS AND METHOD, AND RECORDING MEDIUM

(71) Applicant: SAMSUNG ELECTRONICS CO., LTD., Suwon-si (KR)

(72) Inventors: Dong-jin YANG, Seoul (KR); Hye-sun KIM, Suwon-si (KR); Kyoung-yong LEE, Hwaseong-si (KR)

(73) Assignee: SAMSUNG ELECTRONICS CO., LTD., Suwon-si (KR)

(21) Appl. No.: 14/585,825

(22) Filed: Dec. 30, 2014

(30) Foreign Application Priority Data
Dec. 30, 2013 (KR) 10-2013-0167487

(51) Int. Cl. A61B 5/00; G01R 33/567; A61B 5/024; A61B 6/03; A61B 5/044; A61B 5/0245

(52) U.S. Cl. CPC A61B 5/7289; A61B 5/044; A61B 5/0245; A61B 5/7292; A61B 5/0044; A61B 5/02405; A61B 6/032; G01R 33/5673; A61B 2576/023

(57) ABSTRACT

The image diagnostic method includes acquiring an electrocardiogram (ECG) signal of an object, selecting a scan protocol used to reconfigure a heart image of the object, based on the acquired ECG signal, and providing a user with information used to propose the selected scan protocol.

### CROSS-REFERENCE TO RELATED APPLICATION

This application claims priority from Korean Patent Application No. 10-2013-0167487, filed on Dec. 30, 2013, in the Korean Intellectual Property Office, the disclosure of which is incorporated herein in its entirety by reference.

### BACKGROUND

1. Field

One or more exemplary embodiments relate to an image diagnostic apparatus and method and a recording medium.

2. Description of the Related Art

Examples of high-tech image diagnostic apparatuses, which are used to diagnose various diseases of a human body, include magnetic resonance imaging (MRI) apparatuses, computed tomography (CT) apparatuses, etc.

In order to image an object by using an image diagnostic apparatus, a user needs to select an imaging protocol in consideration of a state of the object. Also, the user needs to consider variables that will occur in imaging. Therefore, it may be difficult for a user to image an object.

In the related art, guidelines for an imaging method necessary to image an object are not provided, and for this reason, a user manually selects a desired imaging method. The quality of an acquired image depends on a user's skill or an imaging method selected by the user.

### SUMMARY

Exemplary embodiments address at least the above problems and/or disadvantages and other disadvantages not described above. Also, the exemplary embodiments are not required to overcome the disadvantages described above, and may not overcome any of the problems described above.

One or more exemplary embodiments include an image diagnostic apparatus and method and a recording medium, which provide guidelines for an imaging method that a user may use to diagnose a disease.

One or more exemplary embodiments include an image diagnostic apparatus and method and a recording medium, which enable a user to easily select an imaging method that a user may use to diagnose a disease.

According to an aspect of an exemplary embodiment, an image diagnostic method includes: acquiring an electrocardiogram (ECG) signal of an object; selecting a scan protocol used to reconfigure a heart image of the object, based on the acquired ECG signal; and providing a user with information used to propose the selected scan protocol.

According to an aspect of an exemplary embodiment, an image diagnostic apparatus includes: an input/output unit that acquires an electrocardiogram (ECG) signal of an object; a controller that selects a scan protocol used to reconfigure a heart image of the object, based on the acquired ECG signal; and an information provider that provides a user with information used to propose the selected scan protocol.

### BRIEF DESCRIPTION OF THE DRAWINGS

The above and/or other aspects will become more apparent by describing certain exemplary embodiments, with reference to the accompanying drawings, in which:

FIG. 1 is a schematic diagram of a computed tomography (CT) system;

FIG. 2 is a diagram illustrating a structure of a CT system according to an exemplary embodiment;

FIG. 3 is a diagram illustrating an image diagnostic system according to an exemplary embodiment;

FIG. 4 is a flowchart for describing an image diagnostic method according to an exemplary embodiment;

FIG. 5 is a flowchart for describing a method in which an image diagnostic apparatus selects a scan protocol for reconfiguring a heart image of an object, according to an exemplary embodiment;

FIG. 6 is a flowchart for describing a method in which the image diagnostic apparatus reconfigures an image of an object based on a determined scan protocol, according to an exemplary embodiment;

FIG. 7 is a diagram illustrating a user interface in which an electrocardiogram signal sample and an acquired electrocardiogram signal are shown as waveforms, according to an exemplary embodiment;

FIG. 8 is a flowchart for describing an image diagnostic method according to an exemplary embodiment;

FIG. 9 is a flowchart for describing a method in which the image diagnostic apparatus sets a reconfiguration section of an image obtained by imaging a heart of an object, according to an exemplary embodiment;

FIG. 10 is a diagram for describing a method in which the image diagnostic apparatus displays a section in which arrhythmia occurs in a user interface, according to an exemplary embodiment; and

FIGS. 11 and 12 are block diagrams of the image diagnostic apparatus according to an exemplary embodiment.

### DETAILED DESCRIPTION

Certain exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

Expressions such as “at least one of,” when preceding a list of elements, modify the entire list of elements and do not modify the individual elements of the list.

When a part “includes” or “comprises” an element, unless there is a particular description contrary thereto, the part can further include other elements, not excluding the other elements. Also, the term ‘unit’ means a software component or hardware components such as a field-programmable gate array (FPGA) or an application-specific integrated circuit (ASIC), and performs a specific function. However, the term ‘unit’ is not limited to software or hardware. The ‘unit’ may be formed so as to be in an addressable storage medium, or may be formed so as to operate one or more processors. Thus, for example, the term ‘unit’ may refer to components such as software components, object-oriented software components, class components, and task components, and may include processes, functions, attributes, procedures, subroutines, segments of program code, drivers, firmware, micro codes, circuits, data, a database, data structures, tables, arrays, or variables. A function provided by the components and ‘units’ may be associated with the smaller number of components and ‘units’, or may be divided into additional components and ‘units’.

Throughout the specification, an “image” may mean multi-dimensional data formed of discrete image elements (e.g., pixels in a two-dimensional (2D) image and voxels in a three-dimensional (3D) image). For example, the image may include a medical image of an object which is captured by a computed tomography (CT) imaging apparatus.

Throughout the specification, a “CT image” may mean an image generated by synthesizing a plurality of X-ray images that are obtained by imaging an object while a CT imaging apparatus rotates about at least one axis with respect to the object.

Throughout the specification, an “object” may include a human, an animal, or a part of a human or animal. For example, the object may include organs such as a liver, heart, womb, brain, breast, abdomen, or the like, or a blood vessel. Also, the object may include a phantom. The phantom is a material having a volume that is very close to a density and effective atomic number of an organism or living thing, and may include a sphere phantom having a characteristic similar to a physical body.

Throughout the specification, a “user” may be, but is not limited to, a medical professional including a doctor, a nurse, a medical laboratory technologist, a medial image expert, and a technician who repairs a medical apparatus.

Since a CT system is capable of providing a cross-sectional image of an object, the CT system may express an inner structure (e.g., an organ such as a kidney, a lung, etc.) of the object without an overlap therebetween, compared to a general X-ray capturing apparatus.

The CT system may obtain a plurality of pieces of image data with a thickness no more than 2 mm for several tens to several hundreds of times per second and then may process the plurality of pieces of image data, so that the CT system may provide a relatively accurate a cross-sectional image of the object. Examples of image reconstruction methods are as below.
A shade surface display (SSD) method: The SSD method is an initial 3D imaging method that displays only voxels having a predetermined Hounsfield Units (HU) value.
A maximum intensity projection (MIP)/minimum intensity projection (MinIP) method: The MIP/MinIP method is a 3D imaging method that displays only voxels having the greatest or smallest HU value from among voxels that construct an image.
A volume rendering (VR) method: The VR method is an imaging method capable of adjusting a color and transmittance of voxels that construct an image, according to interest areas.
A virtual endoscopy method: This method allows an endoscopy observation in a 3D image that is reconstructed by using the VR method or the SSD method.
A multi-planar reformation (MPR) method: The MPR method is used to reconstruct an image into a different cross-sectional image. A user may reconstruct an image in every desired direction.
An editing method: This method involves editing adjacent voxels so as to allow a user to easily observe an interest area in volume rendering.
A voxel of interest (VOI) method: The VOI method displays only a selected area in volume rendering.

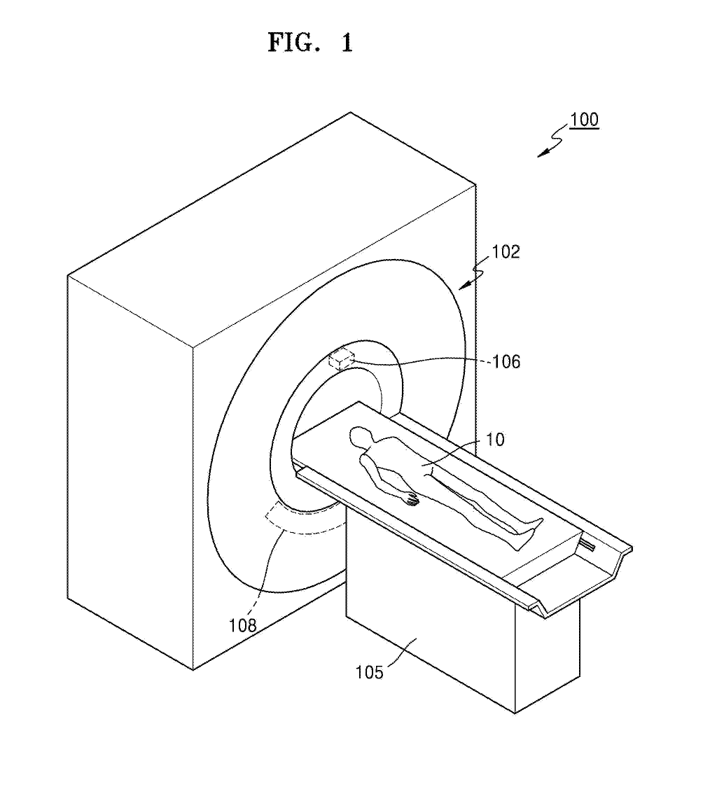

A CT system 100 according to an exemplary embodiment will now be described with reference to FIG. 1. The CT system 100 may include devices having various forms.

FIG. 1 schematically illustrates the CT system 100. Referring to FIG. 1, the CT system 100 may include a gantry 102, a table 105, an X-ray generator 106, and an X-ray detector 108.

The gantry 102 may include the X-ray generator 106 and the X-ray detector 108.

An object 10 may be positioned on the table 105.

The table 105 may move in a predetermined direction (e.g., at least one of up, down, right, and left directions) during a CT imaging procedure. Also, the table 105 may tilt or rotate by a predetermined degree in a predetermined direction.

The gantry 102 may also tilt by a predetermined degree in a predetermined direction.

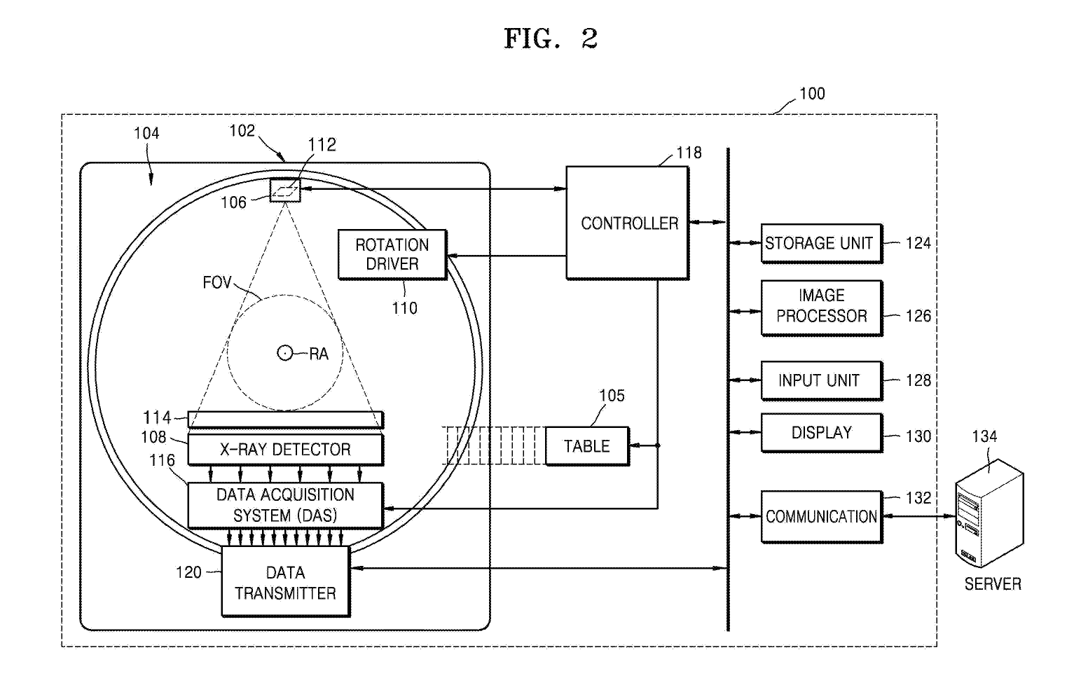

FIG. 2 is a diagram illustrating a structure of the CT system 100.

The CT system 100 may include the gantry 102, the table 105, a controller 118, a storage unit 124, an image processor 126, an input unit 128, a display 130, and a communicator 132.

As described above, the object 10 may be positioned on the table 105. In the present exemplary embodiment, the table 105 may move in a predetermined direction (e.g., at least one of up, down, right and left directions), and movement of the table 105 may be controlled by the controller 118.

The gantry 102 may include a rotating frame 104, the X-ray generator 106, the X-ray detector 108, a rotation driver 110, a data acquisition system (DAS) 116, and a data transmitter 120.

The rotating frame 104 included in the gantry 102 may have a loop shape capable of rotating with respect to a predetermined rotation axis RA. The rotating frame 104 may be disc shaped.

The X-ray generator 106 and the X-ray detector 108 included in the rotating frame 104 may face each other so as to have predetermined field of views (FOVs). The rotating frame 104 may also include an anti-scatter grid 114. The anti-scatter grid 114 may be positioned between the X-ray generator 106 and the X-ray detector 108.

In a medical imaging system, X-ray radiation that reaches a detector (or a photosensitive film) includes attenuated primary radiation that forms a useful image and also includes scattered radiation that deteriorates a quality of an image. In order to transmit the primary radiation and to attenuate the scattered radiation, the anti-scatter grid 114 may be positioned between a patient and the detector (or the photosensitive film).

For example, the anti-scatter grid 114 may be formed by alternately stacking lead foil strips and an interspace material such as a solid polymer material, solid polymer, or a fiber composite material. However, formation of the anti-scatter grid 114 is not limited thereto.

The rotating frame 104 may receive a driving signal from the rotation driver 110 and may rotate the X-ray generator 106 and the X-ray detector 108 at a predetermined rotation speed. The rotating frame 104 may receive the driving signal and power from the rotation driver 110 while the rotating frame 104 contacts the rotation driver 110 via a slip ring (not shown). The rotating frame 104 may receive the driving signal and power from the rotation driver 110 via wireless communication.

The X-ray generator 106 may receive a voltage and current from a power distribution unit (PDU) (not shown) via a slip ring (not shown) and then a high voltage generator (not shown), and then may generate and emit an X-ray. When the high voltage generator applies a predetermined voltage (hereinafter, referred as the tube voltage) to the X-ray generator 106, the X-ray generator 106 may generate X-rays having a plurality of energy spectrums that correspond to the tube voltage.

The X-ray generated by the X-ray generator 106 may have a predetermined form due to a collimator 112 and then may be emitted.

The X-ray detector 108 may be positioned while facing the X-ray generator 106. The X-ray detector 108 may include a plurality of X-ray detecting devices. Each of the plurality of X-ray detecting devices may establish one channel but the exemplary embodiments are not limited thereto.

The X-ray detector 108 may detect the X-ray that is generated by the X-ray generator 106 and that is transmitted through the object 10, and may generate an electrical signal corresponding to intensity of the detected X-ray.

The X-ray detector 108 may include an indirect-type X-ray detector for detecting radiation after converting the radiation into light, and a direct-type X-ray detector for detecting radiation after directly converting the radiation into electric charges. The indirect-type X-ray detector may use a scintillator. The direct-type X-ray detector may use a photon counting detector. The DAS 116 may be connected to the X-ray detector 108. The electrical signal generated by the X-ray detector 108 may be collected by the DAS 116 wirelessly or via a wire. The electrical signal generated by the X-ray detector 108 may be provided to an analog-to-digital converter (not shown) via an amplifier (not shown).

According to a slice thickness or the number of slices, only some of a plurality of data collected by the X-ray detector 108 may be provided to the image processor 126 via the data transmitter 120, or the image processor 126 may select only some of the plurality of data.

The digital signal may be provided to the image processor 126 via the data transmitter 120. The digital signal may be provided wirelessly or via a wire to the image processor 126.

The controller 118 may control an operation of the elements in the CT system 100. For example, the controller 118 may control operations of the table 105, the rotation driver 110, the collimator 112, the DAS 116, the storage unit 124, the image processor 126, the input unit 128, the display 130, the communicator 132, and the like.

The image processor 126 may receive data (e.g., pure data before a processing operation), which is obtained from the DAS 116, via the data transmitter 120, and may perform pre-processing.

The pre-processing may include a process of correcting sensitivity irregularity between channels, a process of correcting a signal loss due to a rapid decrease in signal strength or due to an X-ray absorbing material such as metal, or the like.

Data output from the image processor 126 may be referred to as raw data or projection data. The projection data and image-capturing conditions (e.g., the tube voltage, an image-capturing angle, etc.) during obtainment of the data may be stored together in the storage unit 124.

The projection data may be a group of data values that correspond to the intensity of the X-ray that passes through the object 10. For convenience of description, it is assumed that a group of a plurality of pieces of projection data that are simultaneously obtained from all channels of a same image-capturing degree is referred to as a projection data set.

The storage unit 124 may include at least one storage medium from a flash memory-type storage medium, a hard disk-type storage medium, a multimedia card micro-type storage medium, card-type memories (e.g., an SD card, an XD memory, and the like), Random Access Memory (RAM), Static Random Access Memory (SRAM), Read-Only Memory (ROM), Electrically Erasable Programmable Read-Only Memory (EEPROM), Programmable Read-Only Memory (PROM) magnetic memory, a magnetic disc, and an optical disc.

The image processor 126 may reconstruct a cross-sectional image with respect to the object 10 by using the projection data set. The cross-sectional image may be a 3D image. The image processor 126 may reconstruct the 3D image of the object 10 by using a cone beam reconstruction method or the like, based on the projection data set.

The input unit 128 may receive an external input with respect to an X-ray tomography imaging condition, an image processing condition, or the like. For example, the X-ray tomography imaging condition may include a tube voltage, energy value setting with respect to a plurality of X-rays, selection of an image-capturing protocol, selection of an image reconstruction method, setting of a FOV area, the number of slices, a slice thickness, parameter setting with respect to image post-processing, or the like. The image processing condition may include resolution of an image, attenuation coefficient setting with respect to the image, setting of an image combining ratio, or the like.

The input unit 128 may include a device for receiving a predetermined input from an external source. For example, the input unit 128 may include a microphone, a keyboard, a mouse, a joystick, a touch pad, a touch pen, a voice recognition device, a gesture recognition device, or the like.

The display 130 may display an X-ray tomography image reconstructed by the image processor 126.

Exchanges of data, power, or the like between the aforementioned elements may be performed by using at least one of wired communication, wireless communication, and optical communication.

The communicator 132 may communicate with an external device, an external medical apparatus, etc., via a server 134 or the like.

The communicator 132 may exchange data with a hospital server or other medical apparatuses in a hospital connected via a Picture Archiving and Communication System (PACS). The communicator 132 may perform data communication with the portable device, according to a Digital Imaging and Communications in Medicine (DICOM) standard.

The communicator 132 may transmit and receive data related to diagnosing the object 10, via the network. The communicator 132 may transmit and receive a medical image.

Furthermore, the communicator 132 may receive a diagnosis history or a medical treatment schedule about a patient from the server 134 and may use the diagnosis history or the medical treatment schedule in a clinical diagnosis for the patient. The communicator 132 may perform data communication with the portable device of a user or patient.

The communicator 132 may transmit information about a device error, information about a quality control status, or the like to a system manager or a service manager via the network, and may receive a feedback corresponding to the information.

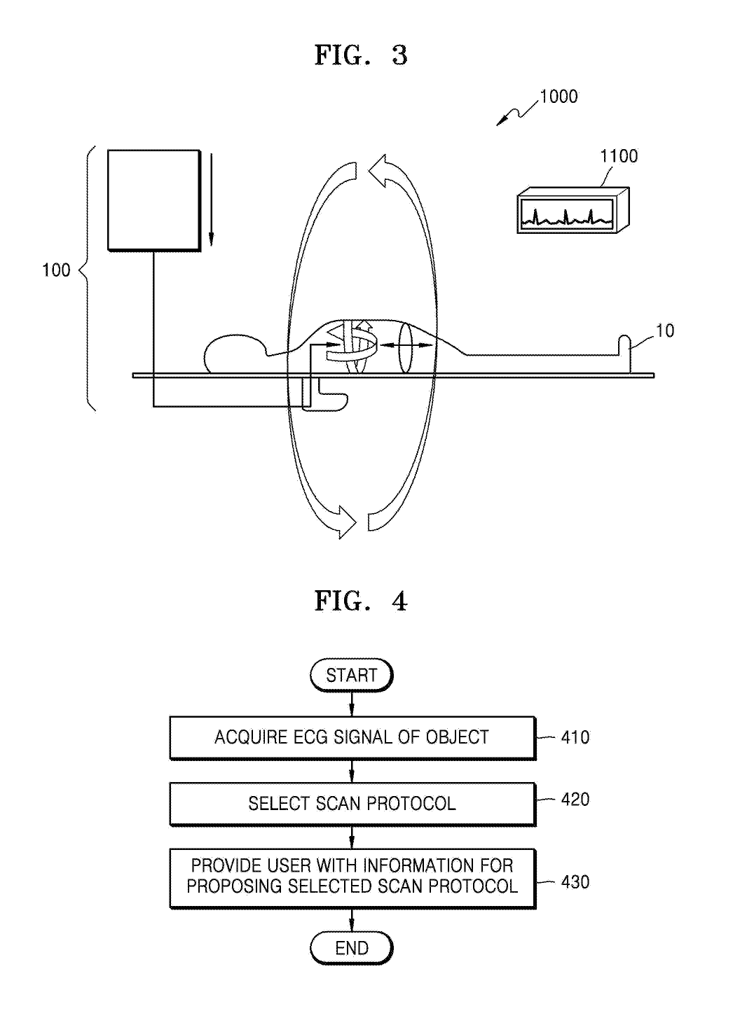

FIG. 3 is a diagram illustrating an image diagnostic system 1000 according to an exemplary embodiment.

The image diagnostic system 1000 of FIG. 3 is illustrated as including only elements associated with the present exemplary embodiment. However, the image diagnostic system 1000 may further include other general-use elements in addition to the elements of FIG. 3.

Referring to FIG. 3, the image diagnostic system 1000 according to an exemplary embodiment may include an image diagnostic apparatus 1100 and a CT system 100.

The image diagnostic apparatus 1100 may provide a user with guidelines for reconfiguring an image of an object 10 which is captured by the CT system 100. The image diagnostic apparatus 1100 may provide the user with scan protocol information suitable for reconfiguring a captured image of the object 10 based on information of the object 10. For example, the image diagnostic apparatus 1100 may provide scan protocol information suitable for reconfiguring a heart image of the object 10 which is captured by the CT system 100, based on an electrocardiogram (ECG) signal of the object 10.

The CT system 100 is merely an example of a system that is used to image the object 10 in an exemplary embodiment, but is not limited thereto. For example, the image diagnostic apparatus 1100 may provide the user with guidelines for reconfiguring an image of the object 10 which is captured by an MRI apparatus.

The image diagnostic apparatus 1100 according to an exemplary embodiment may communicate with another device. For example, the image diagnostic apparatus 1100 may communicate with the CT system 100. The image diagnostic apparatus 1100 may communicate with a device, such as an ECG device, which acquires a bio-signal from the object 10.

The image diagnostic apparatus 1100 may be provided outside the CT system 100 as in FIG. 3, but is not limited thereto. According to another exemplary embodiment, the image diagnostic apparatus 1100 may include the CT system 100.

The CT system 100 may project X-rays onto an object at various angles, and generate an image by reconfiguring an internal cross-sections of the object, based on the projection result.

FIG. 4 is a flowchart for describing an image diagnostic method according to an exemplary embodiment.

In operation 410, the image diagnostic apparatus 1100 acquires an ECG signal of the object 10.

The image diagnostic apparatus 1100 may receive data of the ECG signal of the object 10, as measured by the ECG device. According to another exemplary embodiment, the image diagnostic apparatus 1100 may receive the data of the ECG signal of the object 10 from an external server that stores medical information of the object 10.

The image diagnostic apparatus 1100 may acquire bio-signal information of the object 10, in addition to the ECG signal of the object 10. The bio-signal information is not limited to an electrical signal acquirable from the object 10. For example, the image diagnostic apparatus 1100 may acquire a heart image of the object 10 captured by the CT system 100. In the image diagnostic method according to an exemplary embodiment, a case in which the ECG signal is acquired as the information acquired in operation 410 for acquiring a scan protocol will be described as an example.

In operation 420, the image diagnostic apparatus 1100 selects a scan protocol used to reconfigure a heart image of an object, based on the acquired ECG signal.

Examples of the scan protocol may include a prospective scan protocol, a retrospective scan protocol, and a multi-segment scan protocol. The scan protocols may further include other appropriate scan protocols; however, exemplary embodiments are described with reference to the above examples of the protocols.

The image diagnostic apparatus 1100 may select a scan protocol used to reconfigure a captured image of the object 10, based on at least one of a period of the acquired ECG signal and a heart rate that is calculated based on the acquired ECG signal. The image diagnostic apparatus 1100 may select a scan protocol to be executed automatically, or may recommend the scan protocol to a user as a candidate scan protocol, to be confirmed and/or verified by the user.

In operation 430, the image diagnostic apparatus 1100 may provide a user with information which has been used to propose the selected scan protocol. The image diagnostic apparatus 1100 according to an exemplary embodiment displays a user interface that displays information about the selected scan protocol. For example, the image diagnostic apparatus 1100 may display the selected scan protocol as text data or image data in the user interface.

The image diagnostic apparatus 1100 may display an explanation of a method in which the selected scan protocol reconfigures the captured image of the object 10 and/or a reason substantiating as to why the image diagnostic apparatus 1100 selected a certain scan protocol for the acquired ECG signal.

For example, when the image diagnostic apparatus 1100 selects the prospective scan protocol, the image diagnostic apparatus 1100 may determine the ECG signal acquired from the object 10 as being periodic, and display information, indicating a selection of the prospective scan protocol, in a certain region of the user interface.

The image diagnostic apparatus 1100 according to an exemplary embodiment may provide the user with guidelines for reconfiguring the captured image of the object 10, based on the information acquired from the object 10, thereby aiding the user in easily reconfiguring the captured image of the object 10.

The image diagnostic apparatus 1100 according to an exemplary embodiment may display the acquired ECG signal along with an ECG signal sample corresponding to the selected scan protocol. The sample of the ECG signal corresponding to the selected scan protocol may be information which is pre-stored and is used for the image diagnostic apparatus 1100 to select a scan protocol in comparison with the acquired ECG signal.

For example, the image diagnostic apparatus 1100 may store in advance a first ECG signal sample, a second ECG signal sample, and a third ECG signal sample. When the acquired ECG signal is most similar to the first ECG signal sample, the image diagnostic apparatus 1100 may select the prospective scan protocol as a scan protocol used to reconfigure the heart image of the object 10. When the acquired ECG signal is most similar to the second ECG signal sample, the image diagnostic apparatus 1100 may select the retrospective scan protocol as the scan protocol used to reconfigure the heart image of the object 10. When the acquired ECG signal is most similar to the third ECG signal sample, the image diagnostic apparatus 1100 may select the multi-segment scan protocol as the scan protocol used to reconfigure the heart image of the object 10.

For example, the first ECG signal sample corresponds to an ECG signal having a substantially regular period. The second ECG signal sample corresponds to an ECG signal having an irregular period. The third ECG signal sample corresponds to an ECG signal that has a higher heart rate than an average heart rate.

The image diagnostic apparatus 1100 may differently set an ECG signal sample used to select a scan protocol, based on a disease, age, and gender of the object 10.

The image diagnostic apparatus 1100 according to an exemplary embodiment may provide the user with the acquired ECG signal, ECG signal sample and the selected scan protocol, thereby providing the user with a scan protocol which is suitable for reconfiguring the heart image of the object 10.

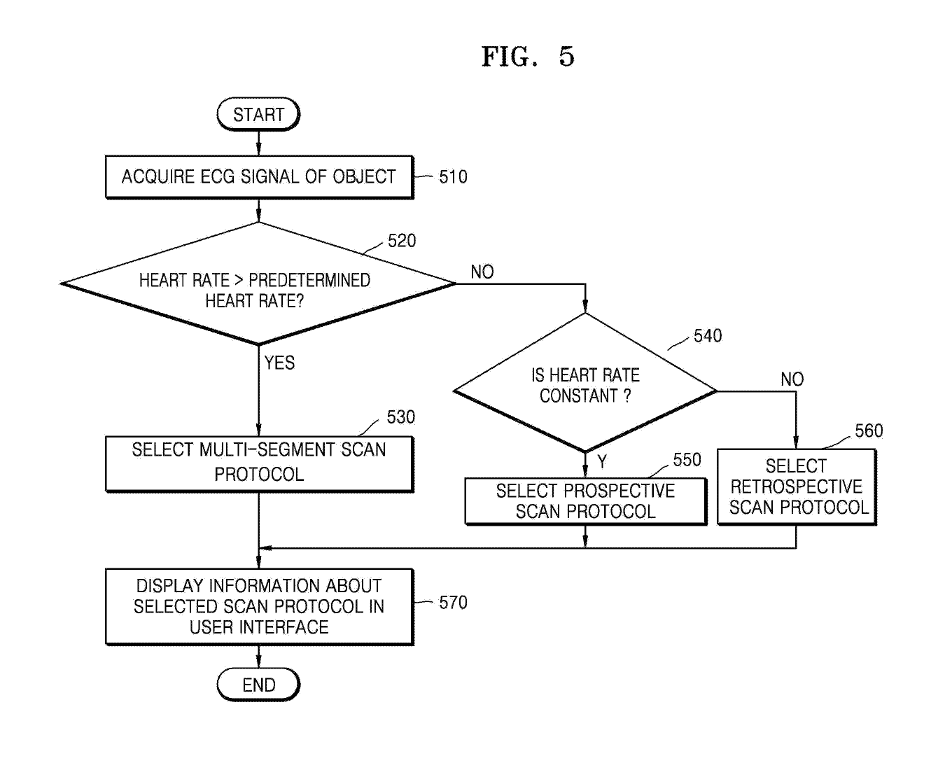

FIG. 5 is a flowchart for describing a method in which the image diagnostic apparatus 1100 selects a scan protocol for reconfiguring a heart image of an object 10, according to an exemplary embodiment.

In operation 510, the image diagnostic apparatus 1100 may acquire an ECG signal of the object 10. The image diagnostic apparatus 1100 may receive data of the ECG signal of the object 10, as measured by the ECG device.

Operation 510 of FIG. 5 may correspond to operation 410 of FIG. 4.

In operation 520, the image diagnostic apparatus 1100 may determine whether a heart rate, which is calculated based on the acquired ECG signal, exceeds a predetermined heart rate.

The image diagnostic apparatus 1100 according to an exemplary embodiment may determine whether to select the multi-segment scan protocol as a protocol used to reconfigure a heart image of the object 10, based on the heart rate which is calculated based on the acquired ECG signal. For example, when a heart rate is high, the multi-segment scan protocol may be used to reconfigure an image of a heart.

For example, when a heart rate which is calculated based on an ECG signal exceeds 100 per minute, the image diagnostic apparatus 1100 may select the multi-segment scan protocol as a protocol used to reconfigure an image of a heart. The heart rate may be an average heart rate which is calculated for a predetermined time based on the ECG signal.

A user may adjust a predetermined heart rate in the image diagnostic apparatus 1100. The user may adjust the predetermined heart rate so as to be suitable for a purpose of reconfiguring the image of the heart.

In operation 530, the image diagnostic apparatus 1100 may select the multi-segment scan protocol as a protocol used to reconfigure the heart image of the object 10. The image diagnostic apparatus 1100 according to an exemplary embodiment may select the multi-segment scan protocol for reconfiguring a captured image of a heart of an object 10 having a heart rate which exceeds the predetermined heart rate.

If a heart rate of the object is determined as not exceeding the predetermined heart rate in operation 520, the image diagnostic apparatus 1100 may determine whether a period of the acquired ECG signal is constant, in operation 540. The image diagnostic apparatus 1100 may determine whether the period of the acquired ECG signal is constant, for reconfiguring a captured image of a heart of an object 10 which is equal to or lower than the predetermined heart rate.

Generally, when a period of an ECG signal is constant, the prospective scan protocol is used, and when the period of the ECG signal is not constant, the retrospective scan protocol is used.

A related art ECG signal analyzing method may be used as a method in which the image diagnostic apparatus 1100 determines whether the period of the ECG signal is constant. A method in which the image diagnostic apparatus 1100 analyzes the ECG signal is not limited and any appropriate method known to those skilled in the art may be used.

In operation 550, the image diagnostic apparatus 1100 may select the prospective scan protocol as a protocol used to reconfigure the heart image of the object 10. The image diagnostic apparatus 1100 according to an exemplary embodiment may select the prospective scan protocol for reconfiguring a captured image of a heart of an object 10 having a constant heart rate.

In operation 560, the image diagnostic apparatus 1100 may select the retrospective scan protocol as a protocol used to reconfigure the heart image of the object 10. The image diagnostic apparatus 1100 according to an exemplary embodiment may select the retrospective scan protocol for reconfiguring a captured image of a heart of an object 10 having an irregular heart rate.

The image diagnostic apparatus 1100 may first determine whether the period of the acquired ECG signal is constant, before calculating the heart rate from the acquired ECG signal. That is, in a method where the image diagnostic apparatus 1100 selects a scan protocol, it is possible to switch the order in which operation 520 and operation 540 are performed.

In operation 570, the image diagnostic apparatus 1100 may display a user interface that displays information about the selected scan protocol on a display included in the image diagnostic apparatus 1100 or on separately provided display.

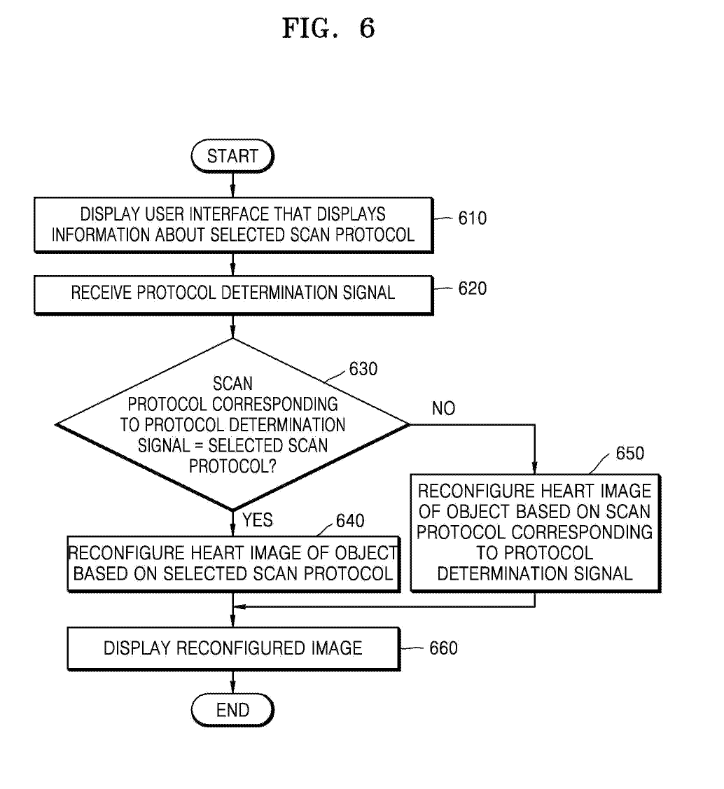

FIG. 6 is a flowchart for describing a method in which the image diagnostic apparatus 1100 reconfigures an image of an object 10 based on a determined scan protocol, according to an exemplary embodiment.

In operation 610, the image diagnostic apparatus 1100 may display a user interface that displays information about a selected scan protocol.

Operation 610 of FIG. 6 may correspond to the above-described operation 410 of FIG. 4.

In operation 620, the image diagnostic apparatus 1100 may receive a protocol determination signal, used to determine a scan protocol, from a user through the user interface. The user may determine the scan protocol based on the information which is displayed in the user interface about the scan protocol.

According to an exemplary embodiment, the user interface may display, as information about a scan protocol, an ECG signal sample for a scan protocol, which are pre-stored and pre-selected, and an ECG signal which is acquired by the image diagnostic apparatus 1100.

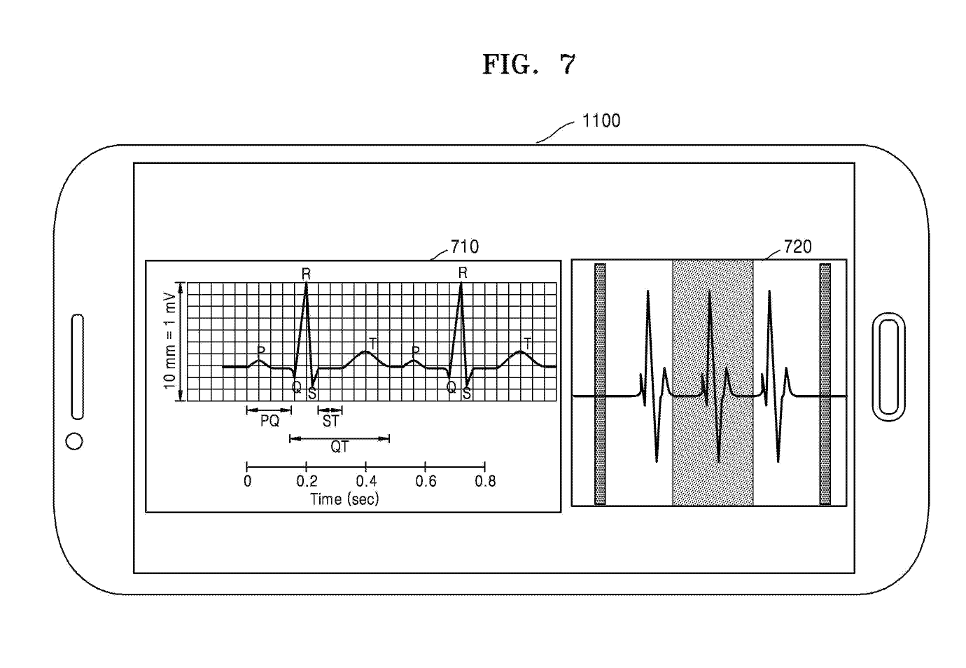

FIG. 7 is a diagram illustrating a user interface in which an ECG signal sample 710 and an acquired ECG signal 720 are shown as waveforms, according to an exemplary embodiment.

Referring to FIG. 7, a user may compare the ECG signal sample 710 and the acquired ECG signal 720, which are displayed in the user interface, to determine whether to reconfigure a heart image of an object 10, by using a scan protocol selected by the image diagnostic apparatus 1100.

The image diagnostic apparatus 1100 may display an explanation facilitating an analysis of a waveform as additional information in the ECG signal sample 710. Statistical data of each section of the ECG signal may be displayed in the ECG signal sample 710. For example, the statistical data may be displayed for each of an RR section, a P-Q section, a Q-T section, and an S-T section. The image diagnostic apparatus 1100 may display information about a statistically used scan protocol depending on a length of each section.

The image diagnostic apparatus 1100 may display information indicating a degree of similarity between the ECG signal sample 710 and the acquired ECG signal 720.

For example, the user may perform setting so that the image diagnostic apparatus 1100 extracts an ECG signal sample having a similarity of about 80% or more with an acquired ECG signal to select a scan protocol. When a similarity of about 90% is calculated as a result that is obtained by analyzing a similarity between waveforms of the ECG signal sample 710 and the acquired ECG signal 720, information indicating a calculated similarity being about 90% may be displayed as additional information along with information of the selected scan protocol, in the user interface.

The user interface of FIG. 7 is merely an example of the present exemplary embodiment, and various user interfaces may be provided. In FIG. 7, a case in which the ECG signal sample 710 and the acquired ECG signal 720 are displayed separately from each other is shown as an example, but the image diagnostic apparatus 1100 may display the ECG signal sample 710 and the acquired ECG signal 720 to be overlapped with each other.

In operation 630, the image diagnostic apparatus 1100 may determine whether a scan protocol corresponding to the protocol determination signal received from a user matches the scan protocol selected by the image diagnostic apparatus 1100. The user may determine a scan protocol, in addition to the scan protocol selected by the image diagnostic apparatus 1100.

The protocol determination signal may include identification information that enables the image diagnostic apparatus 1100 to select one scan protocol among a plurality of selectable scan protocols.

In operation 640, the image diagnostic apparatus 1100 may reconfigure a captured heart image of the object 10 based on the selected scan protocol when the selected scan protocol matches a scan protocol determined by the user, i.e., confirmed or verified by a user.

The image diagnostic apparatus 1100 may be provided independently from the CT system 100, or may include the CT system 100 and operate as an image diagnostic system. When the image diagnostic apparatus 1100 is provided independently from the CT system 100, the image diagnostic apparatus 1100 may transmit a signal, which includes information about a determined scan protocol and a command for reconfiguring the captured heart image of the object 10, to the CT system 100. The CT system 100 may reconfigure the captured heart image of the object 10 according to the determined scan protocol, based on the signal received from the image diagnostic apparatus 1100.

According to another exemplary embodiment, when the image diagnostic apparatus 1100 includes the CT system 100, the image diagnostic apparatus 1100 may reconfigure the captured heart image of the object 10 based on the determined scan protocol.

In operation 650, the image diagnostic apparatus 1100 may reconfigure the captured heart image of the object 10 based on the scan protocol determined by the user when the selected scan protocol does not match the scan protocol determined by the user.

A method, which reconfigures the captured heart image of the object 10 based on the determined scan protocol (operation 650), may correspond to a method that reconfigures the captured heart image of the object 10 based on the selected scan protocol (operation 640).

In operation 660, the image diagnostic apparatus 1100 may display the reconfigured image on the display of the image diagnostic apparatus 1100.

When the image diagnostic apparatus 1100 is provided independently from the CT system 100, the image diagnostic apparatus 1100 may receive reconfigured image information from the CT system 100, and display the received image information.

According to another exemplary embodiment, when the image diagnostic apparatus 1100 includes the CT system 100, the image diagnostic apparatus 1100 may display image information that is reconfigured based on the determined or selected scan protocol.

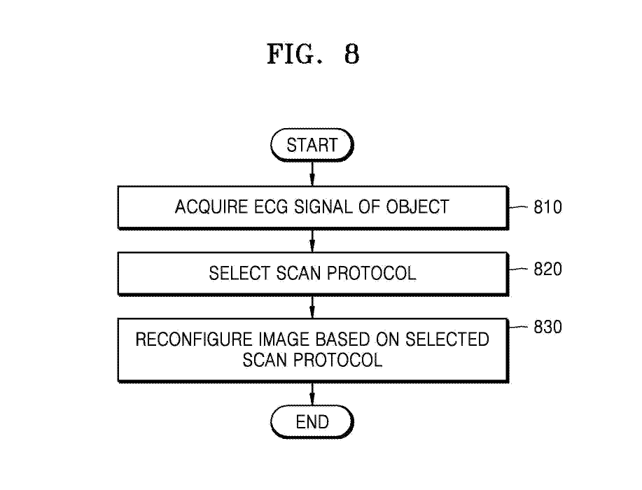

FIG. 8 is a flowchart for describing an image diagnostic method according to an exemplary embodiment.

In operation 810, the image diagnostic apparatus 1100 may acquire an ECG signal of an object 10.

The image diagnostic apparatus 1100 may receive data of the ECG signal of the object 10, as measured by the ECG device, or may receive the data of the ECG signal of the object 10 from an external server that stores medical information of the object 10.

Operation 810 of FIG. 8 may correspond to the above-described operation 410 of FIG. 4.

In operation 820, the image diagnostic apparatus 1100 may select a scan protocol to reconfigure a captured heart image of the object 10, based on the acquired ECG signal.

According to an exemplary embodiment, the image diagnostic apparatus 1100 may select a scan protocol to reconfigure a captured image of the object 10, based on at least one of a period of the acquired ECG signal and a heart rate that is calculated based on the acquired ECG signal.

Operation 820 of FIG. 8 may correspond to the above-described operation 420 of FIG. 4.

In operation 830, the image diagnostic apparatus 1100 may reconfigure a captured heart image of the object 10 based on the selected scan protocol.

According to an exemplary embodiment, the image diagnostic apparatus 1100 may reconfigure a captured heart image of the object 10 based on the selected scan protocol even without receiving a user input signal.

The image diagnostic apparatus 1100 may be provided independently from the CT system 100, or may be included in the CT system 100 and operate as the image diagnostic system. When the image diagnostic apparatus 1100 is provided independently from the CT system 100, the image diagnostic apparatus 1100 may transmit a signal, which includes information about the selected scan protocol and a command for reconfiguring the captured heart image of the object 10, to the CT system 100 via a wired/wireless connection. The CT system 100 may reconfigure the captured heart image of the object 10 according to the selected scan protocol, based on the signal received from the image diagnostic apparatus 1100.

According to another exemplary embodiment, when the image diagnostic apparatus 1100 is included in the CT system 100, the image diagnostic apparatus 1100 may reconfigure the captured heart image of the object 10 based on the selected scan protocol.

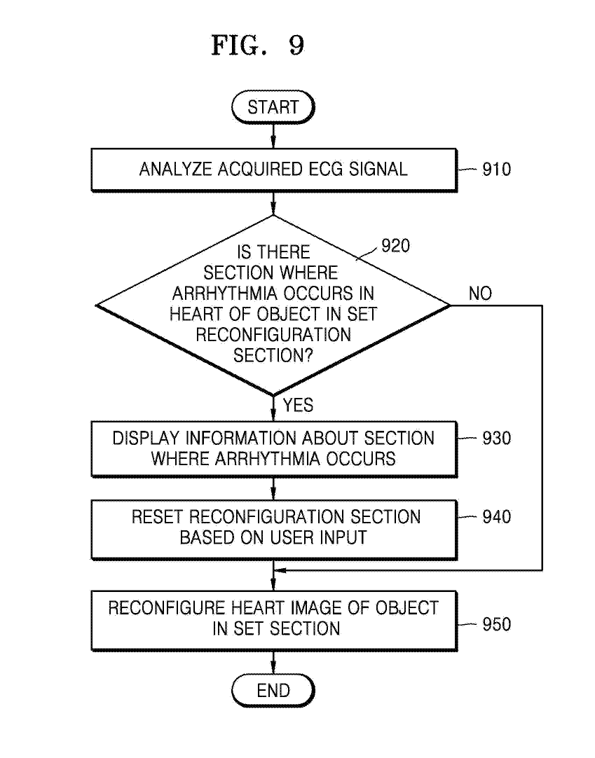

FIG. 9 is a flowchart for describing a method in which the image diagnostic apparatus 1100 sets a reconfiguration section of a captured heart image of an object 10, according to an exemplary embodiment.

In operation 910, the image diagnostic apparatus 1100 may analyze an ECG signal acquired from the object 10.

The image diagnostic apparatus 1100 may acquire the heart image of the object 10 along with the ECG signal. According to another exemplary embodiment, the image diagnostic apparatus 1100 may analyze the captured heart image of the object 10.

In operation 920, the image diagnostic apparatus 1100 may determine whether there is a section where arrhythmia occurs, in the acquired ECG signal.

According to an exemplary embodiment, the image diagnostic apparatus 1100 may store information about an ECG signal of when the arrhythmia occurs. For example, the image diagnostic apparatus 1100 may store a sample waveform of the ECG signal of when arrhythmia occurs. The image diagnostic apparatus 1100 may compare the stored sample waveform and a waveform of an acquired ECG signal to determine whether there is a section where arrhythmia occurs in a section of the acquired ECG signal.

The method, which determines whether arrhythmia occurs based on an ECG signal, is merely an example, and is not limited thereto. For example, the image diagnostic apparatus 1100 may analyze the captured heart image of the object 10 to determine whether there is a section where arrhythmia occurs.

In operation 930, the image diagnostic apparatus 1100 may display information about a section, where arrhythmia occurs, in the user interface.

According to an exemplary embodiment, when there is arrhythmia-occurring section as a result that is obtained by determining whether arrhythmia occurs in a section of the ECG signal acquired by the image diagnostic apparatus 1100, the image diagnostic apparatus 1100 may display a section where arrhythmia occurs in the section of the acquired ECG signal, in the user interface.

In operation 940, the image diagnostic apparatus 1100 may reset a section for reconfiguring the captured heart image based on a user input. When the user inputs a signal for commanding a section of the acquired ECG signal to be changed, the image diagnostic apparatus 1100 may reset a section, to exclude the section where arrhythmia occurs in the section of the acquired ECG signal.

On the other hand, when the user does not input a command to change the section of the acquired ECG signal, the image diagnostic apparatus 1100 does not exclude the section where arrhythmia occurs in the section of the acquired ECG signal.

In operation 950, the image diagnostic apparatus 1100 may reconfigure the heart image of the object 10 in the reset section.

According to an exemplary embodiment, when the image diagnostic apparatus 1100 is not provided independently from the CT system 100, the image diagnostic apparatus 1100 may transmit a signal, which includes information about a selected scan protocol, information about a set section, and a command for reconfiguring the captured heart image of the object 10, to the CT system 100. The CT system 100 may reconfigure the captured heart image of the object 10 according to the selected scan protocol, based on the signal received from the image diagnostic apparatus 1100.

According to another exemplary embodiment, when the image diagnostic apparatus 1100 includes the CT system 100, the image diagnostic apparatus 1100 may reconfigure the captured heart image of the object 10 in a section that is set based on the selected scan protocol.

According to an exemplary embodiment, the image diagnostic apparatus 1100 may provide the user with signal section information which is used to propose an ECG signal section for reconfiguring a heart of an object in an entire section of an acquired ECG signal. For example, the image diagnostic apparatus 1100 may provide the user with, as the signal section information, a section or sections, excluding a section or sections where arrhythmia occurs, in an entire section of the acquired ECG signal.

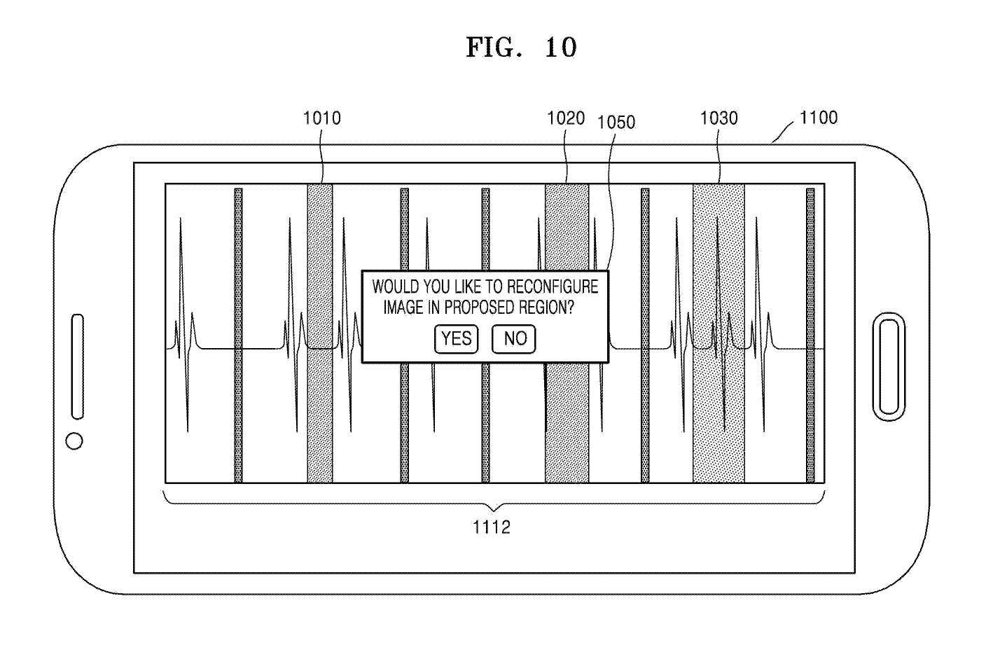

FIG. 10 is a diagram for describing a method in which the image diagnostic apparatus 1100 displays a section in which arrhythmia occurs in a user interface, according to an exemplary embodiment. Referring to FIG. 10, the image diagnostic apparatus 1100 may display that arrhythmia occurs in three parts 1010, 1020 and 1030 illustrated as box blocks, of an entire portion 1112 of an ECG signal displayed in the user interface.

According to an exemplary embodiment, the image diagnostic apparatus 1100 may recommend a section which does not include an arrhythmic section, where arrhythmia occurs, as a section for reconfiguring the captured heart image of the object 10, to the user.

In FIG. 10, the image diagnostic apparatus 1100 may display a conversation box block 1050 for checking whether to accept the section that is recommended to the user for reconfiguring the captured heart image. When the user selects “yes” from the conversation box block 1050, the image diagnostic apparatus 1100 may newly set a first section to not include a section where arrhythmia occurs in the section of the acquired ECG signal. On the other hand, when the user selects “no” from the conversation box block 1050, the image diagnostic apparatus 1100 may immediately reconfigure the captured heart image without any change in the section of the acquired ECG signal.

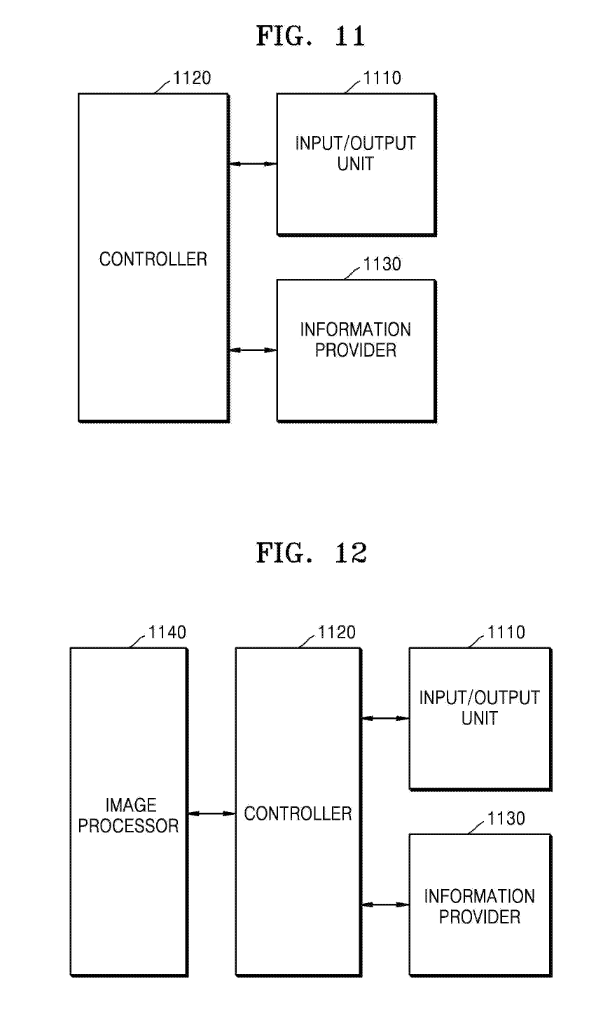

FIGS. 11 and 12 are block diagrams of the image diagnostic apparatus 1100 according to an exemplary embodiment.

As illustrated in FIG. 11, the image diagnostic apparatus 1100 according to an exemplary embodiment may include an input/output unit 1110, a controller 1120, and an information provider 1130. However, the image diagnostic apparatus 1100 may include other elements in addition to the illustrated elements or may include less elements than the illustrated elements.

For example, as illustrated in FIG. 12, the image diagnostic apparatus 1100 may further include an image processor 1140.

The input/output unit 1110 may include one or more elements that enable communication between the image diagnostic apparatus 1100 and another device or another system. For example, the input/output unit 1110 may perform Bluetooth communication, Bluetooth low energy (BLE) communication, short-distance wireless communication, and the like.

The input/output unit 1110 communicates with another device or another system to acquire information about the object 10. For example, the input/output unit 1110 may acquire the ECG signal of the object 10. According to an exemplary embodiment, the input/output unit 1110 may receive data of the ECG signal of the object 10, as measured by the ECG device, or may receive the data of the ECG signal of the object 10 from an external server that stores medical information of the object 10.

The input/output unit 1110 may acquire bio-signal information of the object 10, in addition to the ECG signal of the object 10. The bio-signal information acquired by the input/output unit 1110 is not limited to an electrical signal acquirable from the object 10. For example, the input/output unit 1110 may acquire a heart image of the object 10 captured by the CT system 100.

The controller 1120 selects a scan protocol used to reconfigure a heart image of an object, based on the acquired ECG signal.

The controller 1120 may select a scan protocol used to reconfigure a captured image of the object 10, based on at least one of a period of the acquired ECG signal and a heart rate that is calculated based on the acquired ECG signal.

For example, the controller 1120 may determine whether a heart rate, which is calculated based on the acquired ECG signal, exceeds a predetermined heart rate. The controller 1120 according to an exemplary embodiment may determine whether to select the multi-segment scan protocol as a protocol used to reconfigure a heart image of the object 10, based on the heart rate which is calculated based on the acquired ECG signal.

The controller 1120 may determine whether a period of the acquired ECG signal is constant. The controller 1120 may determine whether the period of the acquired ECG signal is constant, for reconfiguring a captured image of a heart of an object 10 which is equal to or lower than the predetermined heart rate.

For example, when a period of an ECG signal is constant, the prospective scan protocol is used, and when the period of the ECG signal is not constant, the retrospective scan protocol is used.

The controller 1120 according to an exemplary embodiment may first determine whether the period of the acquired ECG signal is constant, before determining for the heart rate calculated from the acquired ECG signal.

The information provider 1130 may include a display panel (not shown) that displays a screen corresponding to image data, and display the screen in the display panel.

The information provider 1130 may be configured to include a touch screen, for example, a touch pad coupled to the display panel (not shown). In this case, the information provider 1130 may output the touch screen onto the display panel, and when a command is input through the touch screen, the touch pad may detect the command.

For example, a user interface screen may be output onto the display panel. When the user touches a certain position of the user interface screen, the touch pad detects the touched position. The information about the touched position may be transferred to the controller 1120.

The information provider 1130 according to an exemplary embodiment may provide the user with information used to propose the selected scan protocol to the user. For example, the information provider 1130 may display a user interface that displays information about the selected scan protocol. The information provider 1130 may display an explanation of a method by which the selected scan protocol reconfigures the captured image of the object 10. The information provider 1130 may display the reason or information indicating as to why a certain scan protocol has been selected, i.e., recommended, for the acquired ECG signal.

The information provider 1130 according to an exemplary embodiment may display the acquired ECG signal along with a sample of an ECG signal corresponding to the selected scan protocol. The sample of the ECG signal corresponding to the selected scan protocol with the selected scan protocol applied thereto may be included in a database that is used by the image diagnostic apparatus 1100 to select a scan protocol in comparison with the acquired ECG signal.

The information provider 1130 according to an exemplary embodiment may display pre-stored scan protocol information about an ECG signal of which a similarity with the acquired ECG signal is equal to or greater than a predetermined value.

The information provider 1130 may receive a protocol determination signal, used to determine a scan protocol, from the user.

The information provider 1130 may provide the user with signal section information which is used to propose an ECG signal section for reconfiguring a heart of an object in a whole section of an acquired ECG signal. For example, the information provider 1130 may provide the user with, as the signal section information, a section except a section where arrhythmia occurs in a whole section of the acquired ECG signal.

The image processor 1140 may reconfigure the heart image of the object 10 based on a scan protocol corresponding to the protocol determination signal received from the user. When the scan protocol corresponding to the received protocol determination signal is the same as the selected protocol, the image processor 1140 may reconfigure the heart image of the object 10 based on the selected scan protocol. On the other hand, when the scan protocol corresponding to the received protocol determination signal differs from the selected protocol, the image processor 1140 may reconfigure the heart image of the object 10 based on the scan protocol corresponding to the protocol determination signal. The image processor 1140 may set a reconfiguration section for the acquired ECG signal. When the set reconfiguration section includes a first section where arrhythmia occurs in the heart of the object 10, the image processor 1140 may control the image provider 1130 to display information about the first section.

The exemplary embodiments may be written as computer programs and may be implemented in general-use digital computers that execute the programs using a computer-readable recording medium.

Examples of the computer-readable recording medium include magnetic storage media (e.g., ROM, floppy disks, hard disks, etc.), optical recording media (e.g., CD-ROMs, or DVDs), etc.

The foregoing exemplary embodiments and advantages are merely exemplary and are not limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An image diagnostic method comprising:
acquiring an electrocardiogram (ECG) signal of an object;
selecting a scan protocol to reconfigure a heart image of the object, based on the acquired ECG signal; and
providing a user with information which was used to propose the selected scan protocol.

2. The image diagnostic method of claim 1, wherein the selecting the scan protocol comprises:
selecting the scan protocol, based on at least one of a period of the acquired ECG signal and a heart rate of the object.

3. The image diagnostic method of claim 1, wherein the scan protocol comprises one of a prospective scan protocol, a retrospective scan protocol, and a multi-segment scan protocol.

4. The image diagnostic method of claim 1, wherein the providing the information comprises:
displaying a user interface configured to display the acquired ECG signal together with information about the selected scan protocol.

5. The image diagnostic method of claim 1, further comprising:
receiving a protocol determination signal, to designate the scan protocol, from the user; and
reconfiguring the heart image of the object, based on the designated scan protocol corresponding to the received protocol determination signal.

6. The image diagnostic method of claim 5, wherein the reconfiguring the heart image comprises:
reconfiguring the heart image of the object, based on the selected scan protocol, in response to the designated scan protocol being the same as the selected scan protocol; and
reconfiguring the heart image of the object, based on the designated scan protocol, in response to the designated scan protocol being different from the selected scan protocol.

7. The image diagnostic method of claim 5, wherein the reconfiguring the heart image comprises:
setting a reconfiguration section for the acquired ECG signal;
determining that the reconfiguration section includes a first section where arrhythmia occurs in a heart of the object; and
providing the user with information about the first section.

8. The image diagnostic method of claim 5, wherein the reconfiguring the heart image comprises:
reconfiguring the heart image in one or more sections, by excluding an arrhythmic section, in an entire section of the acquired ECG signal,
wherein the arrhythmic section is a section in which arrhythmia occurs in a heart of the object.

9. The image diagnostic method of claim 5, wherein the reconfiguring the heart image comprises:
providing the user with signal section information, which is used to propose an ECG signal section for reconfiguring the heart image of the object in an entire section of an acquired ECG signal.

10. The image diagnostic method of claim 1, wherein the providing the information comprises:
displaying pre-stored scan protocol information about an ECG signal in which a similarity with the acquired ECG signal is equal to or greater than a predetermined value.

11. An image diagnostic apparatus comprising:
an input/output unit configured to acquire an electrocardiogram (ECG) signal of an object;
a controller configured to select a scan protocol to reconfigure a heart image of the object, based on the acquired ECG signal; and
an information provider configured to provide a user with information which was used to propose the selected scan protocol.

12. The image diagnostic apparatus of claim 11, wherein the controller is configured to select the scan protocol, based on at least one of a period of the acquired ECG signal and a heart rate of the object.

13. The image diagnostic apparatus of claim 11, wherein the scan protocol comprises one of a prospective scan protocol, a retrospective scan protocol, and a multi-segment scan protocol.

14. The image diagnostic apparatus of claim 11, wherein the information provider is configured to display a user interface configured to display the acquired ECG signal together with information about the selected scan protocol.

15. The image diagnostic apparatus of claim 11, wherein the information provider is configured to receive a protocol determination signal, to designate the scan protocol, from the user, and
the image diagnostic apparatus further comprises an image processor configured to reconfigure the heart image of the object, based on the designated scan protocol corresponding to the received protocol determination signal.

16. The image diagnostic apparatus of claim 15, wherein the image processor is configured to reconfigure the heart image of the object, based on the selected scan protocol, in response to the designated scan protocol being the same as the selected scan protocol, and reconfigure the heart image of the object, based on the designated scan protocol, in response to the designated scan protocol being different from the selected scan protocol.

17. The image diagnostic apparatus of claim 15, wherein the image processor is configured to set a reconfiguration section for the acquired ECG signal, and
when the set reconfiguration section includes a first section where arrhythmia occurs in a heart of the object, the image processor is configured to perform control so that information about the first section is displayed by the information provider.

18. The image diagnostic apparatus of claim 15, wherein the image processor is configured to reconfigure an image in one or more sections, by excluding an arrhythmic section, in an entire section of the acquired ECG signal, and
the arrhythmic section is a section in which arrhythmia occurs in a heart of the object.

19. The image diagnostic apparatus of claim 15, wherein the information provider is configured to provide the user with signal section information, which is used to propose an ECG signal section for reconfiguring a heart of the object in an entire section of an acquired ECG signal.

20. The image diagnostic apparatus of claim 11, wherein the information provider is configured to display pre-stored scan protocol information about an ECG signal of which a similarity with the acquired ECG signal is equal to or greater than a predetermined value.

21. A non-transitory computer-readable storage medium storing a program which, when executed by a computer, causes the computer to execute the method of claim 1.
